(19) United States
(12) Patent Application Publication
(10) Pub. No.: US 2005/0040339 A1
(43) Pub. Date: Feb. 24, 2005

(54) Method of irradiating frozen material

(75) Inventors: Ruth M. Deady Garcia, Buffalo Grove, IL (US); Anthony Harris, Villa Park, IL (US); Betty Howard, Mt. Prospect, IL (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 10/918,868

(22) Filed: Aug. 16, 2004

Related U.S. Application Data
(60) Provisional application No. 60/497,255, filed on Aug. 22, 2003.

(51) Int. Cl. G01T 1/02

(52) U.S. Cl. 250/472.1

(57) ABSTRACT

The present invention relates to a method of determining a dose of radiation applied to a frozen material comprising the steps of: (a) irradiating a frozen material in a container; (b) determining an applied dose of radiation applied to a first location on the container; and (c) determining an absorbed dose of radiation for the frozen material at a second location within the container in accordance with predetermined data relating to the container.

### FIELD OF THE INVENTION

The present invention relates to methods of irradiating articles, and more particularly to a method of irradiating materials to preserve and decontaminate the same.

### BACKGROUND OF THE INVENTION

An allograft is tissue transferred between two genetically different individuals of the same species. The tissue is typically preserved after removal from a donor by freezing. The number of musculoskeletal allografts occurring each year is dramatically increasing. With the increase in the number of allografts being performed, new concerns about the sterility of the grafts, i.e., tissue, have arisen. Maintenance of sterility is a major concern whether a graft is fresh or preserved.

Historically, tissue specimens were processed with aseptic techniques, (to prevent the introduction of additional contamination), or by terminal sterilization methods. Soaking in antibacterial and antifungal solutions may be used in addition to aseptic recovery to further reduce any micro-flora normally associated with tissue specimens. Exposure to ethylene oxide (EO) gas has been used as a tissue sterilization method, but it has its drawbacks. Ethylene oxide leaves chemical residuals on the tissue specimens that can cause inflammation upon implantation. In addition, the ethylene oxide gas may not penetrate the tissue sufficiently to address non-surface contamination.

A modern processing methodology for terminal sterilization of tissue after freezing is gamma irradiation. Gamma irradiation at doses less than 20 kGy is very effective at killing bacteria, and if done at temperatures of −20° C. to −147° C., damage to biological and physical functions of the tissue is minimized. Freezing in conjunction with gamma irradiation is thus an ideal way for the processing, preservation, and sterilization of tissue.

One of the primary challenges of irradiating a frozen tissue sample is determining the dose of radiation actually received by the frozen tissue sample. Most commonly used dose-measuring or dosimetry methods are influenced by temperature. In this respect, temperature can influence a dosimeter reading resulting in a less accurate or skewed dose analysis. Moreover, placement of a dosimeter within frozen tissue is not practical. Although temperature correction factors are available in current literature, the irradiation process is such that the temperature is not constant throughout the entire process, making the application of a single correction difficult.

Specimen density is also a challenge for the determination of irradiation dose applied. Gamma rays emitted from cobalt (60) (the isotope typically used in industrial and in some medical applications) exhibit deep penetration at 1.17 MeV and 1.33 MeV levels. However, the energies of the gamma rays dissipate as the gamma rays pass through dense material. To maintain the tissue specimens at a low temperature, frozen tissue specimens are generally packed in dry ice where the dry ice has a density of approximately 0.47 g/cm³. As a result, concerns exist that a desired dose of radiation may not be achieved in a tissue sample that is disposed within the dry ice within a carton.

The present invention overcomes these and other problems and provides a method of determining the dose of radiation applied to a frozen material.

### SUMMARY OF THE INVENTION

In accordance with a preferred embodiment of the present invention, there is provided a method of determining a dose of radiation applied to a frozen material comprising the steps of:

(a) irradiating a frozen material in a container;

(b) determining an applied dose of radiation applied to a first location on the container; and

(c) determining an absorbed dose of radiation for the frozen material at a second location within the container in accordance with predetermined data relating to the container.

An advantage of the present invention is a method of determining the dose of radiation applied to a frozen tissue sample.

Another advantage of the present invention is a method as described above that abrogates the skewing effects of low temperature environments on dosimetry measurements.

Another advantage of the present invention is a method as described above wherein the integrity of the sterile environment of the tissue sample is not affected.

Yet another advantage of the present invention is a rapid, convenient, standardized method of measuring the dose of radiation applied to a temperature-compromised material.

A still further advantage of the present invention is a method as described above that has a high degree of accuracy.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a perspective view of a system for irradiating frozen material.

### DETAILED DESCRIPTION OF PREFERRED EMBODIMENT

The present invention relates to a method of determining a dose of radiation applied to a frozen material. The present invention is particularly applicable to determining the dose of radiation applied to frozen tissue used in medical procedures, and will be described with particular reference thereto. However, as will be appreciated by those skilled in the art, the present invention is applicable to determining the dose of radiation applied to other frozen materials, such as by way of example and not limitation, frozen foods and frozen serum.

Broadly stated, the present invention relates to a method of determining a dose of radiation applied to a frozen material comprising the steps of:

(a) irradiating a frozen material in a container;

(b) determining an applied dose of radiation applied to a specific location on the exterior of the container; and

(c) determining an absorbed dose of radiation for the frozen material within the container in accordance with predetermined data relating to the container.

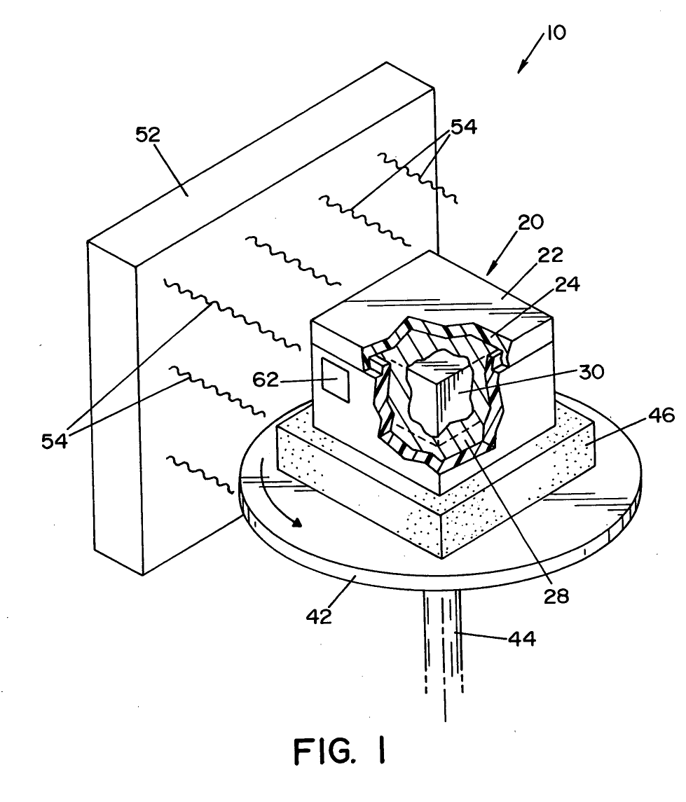

Referring now to the drawings wherein the showings are for the purpose of illustrating a preferred embodiment of the invention only, and not for the purpose of limiting same, FIG. 1 shows a process 10 for irradiating a container 20 containing a frozen target material 30. In the embodiment shown, container 20 is rotatable on a turntable 42 that is rotatable about a shaft 44, as illustrated in FIG. 1. A low density pad 46 supports container 20 on turntable 42. Turntable 42 is disposed adjacent to a source of radiation 52 that emits gamma rays 54. In the embodiment shown, a source of radiation 52 is schematically illustrated as a gamma source. It will of course be appreciated that other forms of radiation, such as e-beam radiation, may also be used.

Container 20 is preferably an insulated structure. In the embodiment shown, container 20 has an outer shell 22 and an insulating lining 24. Centrally disposed within container 20 is a frozen target material, designated 30 in the drawing. In a preferred embodiment, the frozen target material is a tissue sample. A layer of dry ice 28 surrounds the tissue sample. The dry ice is disposed between the tissue sample and insulating lining 24. At a predetermined location on the exterior of the package, a dosimeter is disposed. In the embodiment shown, a dosimeter 62 is disposed on a surface of container 20.

In accordance with the present invention, container 20 is exposed to source 52 of radiation for a predetermined amount of time to effect a determined dose of radiation at dosimeter 62. As will be appreciated by those skilled in the art, the amount of radiation applied to container 20 is based upon the distance of the gamma source from container 20 as well as the exposure time of container 20 to source 52. Dosimeter 62 on container 20 provides an indication of the amount of radiation applied to container 20 at the location of dosimeter 62. According to the present invention, the absorbed dose of radiation on frozen target material 30 within container 20 is determined based upon the absorbed dose of radiation detected by dosimeter 62.

More specifically, the dose of radiation applied to frozen target material 30 within container 20 is determined in accordance with a predetermined mathematical ratio. The ratio is preferably determined through testing container 20 using a surrogate (substitute) material instead of dry ice to surround frozen target material 30. Through testing of container 20 containing a surrogate material (not shown), a ratio of radiation dosage between the outer position of dosimeter 62 and target material 30 can be established. Once such a ratio is determined, the dose of radiation applied to frozen target material 30 is based upon the dose reading of dosimeter 62, without the need for dosimeters within container 20. It will of course be appreciated that a data set could be established instead of a fixed ratio to correlate radiation of an actual frozen material within container 20. The present invention shall now be further described with respect to the method of establishing the data set.

Conventional dosimeters are used to determine absorbed dose values on and within container 20. Dosimeter calibrations are conducted at or about ambient temperatures, i.e., about 25° C. The dosimeters are placed external to the container holding the target material, i.e., frozen tissue samples. In this respect, the surface of the carton preferably remains between 0° C. to 25° C., a temperature range at which little or no temperature correction is required for the dosimeter used in testing. Like, insulated cartons having the same dimensions and physical properties are used for all experiments. Containers 20 are preferably insulated.

A surrogate material, having a density comparable to that of dry ice, is used in determining the mathematical relationship between a radiation dose received at dosimeter 62 on container 20 and the radiation dose received at the geometric center, i.e., the interior, of container 20. It is generally known that the “geometric center” of a homogeneous, three-dimensional structure represents the greatest density challenge and is generally the location of the low dose zone.

It is hypothesized that a numeric ratio can be established between an external position of dosimeter 62 and the central position of container 20 when an ambient temperature “surrogate” material is used in place of dry ice to surround target material 30. The use of such a “surrogate” material, it is believed, is analogous to a real life situation in which a frozen tissue sample is surrounded by dry ice. An additional test is performed using biological indicators to confirm that bacterial kill is achieved based on the calculated dose ratio.

Broadly stated, the test methodology for establishing the numeric ratio includes the following steps:

1. selection of an insulated container 20 of predetermined size and shape;

2. determining the insulating properties of container 20 when container 20 contains dry ice;

3. determining a substitute (surrogate) material to be used in place of dry ice during irradiation testing;

4. establishing delivered dose ratio for container 20; and

5. conducting biological testing to verify the validity of the established dose ratio.

In accordance with the present invention, a standard sized, insulated container 20, appropriate for packaging, transporting and processing of low temperature tissue samples 30 is selected. In a preferred embodiment, a Polyfoam Packer Corporation 22″×14.5″×17.5″ carton is used. Container 20 is selected primarily because of its insulation properties.

The insulating properties of container 20 are evaluated to confirm that dosimeter(s) 62 placed on an external surface of container 20, when container 20 contains dry ice and frozen material, remains at between 0° C. to 25° C. (a temperature range at which little or no temperature correction is required for a dosimeter used in testing).

To verify that the external surface temperature of container 20 remains above 0° C., a Digi-Sense scanning thermometer is used for data collection. Container 20 is filled with dry ice and is then sealed. Thermocouples (not shown) are used to monitor the ambient room temperature, and dosimeters (not shown) are placed on the external surface of container 20.

Temperature data is collected for 1,279 measurements with measurements taken every three minutes. The external temperature of container 20 is monitored for approximately 64 hours to determine if the external surface temperature of container 20 remains above 0° C. Data show that the external surface temperature of container 20 remains between 10° C. and 15° C., establishing that container 20 has sufficient insulative properties such that the temperature of the frozen target material (frozen tissue sample) and dry ice within container 20 will not affect the reading of dosimeter(s) 62 on the surface of container 20.

Once a container 20, having the necessary insulating properties, is selected, a surrogate material is selected and tested to determine if the surrogate material mimics the physical properties of dry ice, i.e., with respect to irradiation. In one embodiment of the present invention, dry dog food is used as the surrogate material. It is contemplated that other materials that mimic the physical properties of dry ice, i.e., with respect to irradiation, may also be used as the surrogate material.

Four separate experiments are conducted to establish a numeric dose ratio value for container 20 between a center position within the insulated carton and an external monitoring position on the surface of carton.

Experiment A is performed with a plurality of testing dosimeters disposed within container 20 and on the external surfaces of container 20. Container 20 is filled with the surrogate material. No target material is within container 20 during this test. Container 20 is irradiated with gamma rays and the absorbed dose readings of the dosimeters are determined. Experiment A is performed to create a baseline characterization to obtain a dose absorption profile of container 20 and the surrogate material at ambient temperatures.

Experiment B is performed with container 20 containing dry ice. Testing dosimeters are located on the external surface of container 20 and within container 20, some preferably at the centermost location. Since dosimeters are temperature sensitive, and the temperature of dry ice typically ranges between −80° C. to −147° C., well outside the normal operating range of most dosimeters, a battery-powered heater is provided with a dosimeter within the container (and within the dry ice) at a location in the center of the container.

The battery-powered heater is designed with an internal non-electrical thermostat. Three separate tests are performed on the dosimeter/heater assembly to determine if the temperature of the dosimeter remains at an acceptable operating level. The data show that for each test, the temperature remained at temperatures between 21° C. to 29° C., well within the operating temperature range of the dosimeters. To prevent the dry ice from affecting the readings of the testing dosimeter within container 20, the battery-powered heater is positioned with the dosimeter(s) within container 20 and warms the same.

The foregoing experiments illustrate that the presence of the activated heater overcomes the chilling effects of the dry ice and maintains the testing dosimeter within the dry ice at a useable operating temperature. Container 20 is irradiated using gamma radiation and the radiation dose absorbed on the external surface of container 20 as well as within container 20 are determined. The results of experiment B establish a numeric ratio between the external dose (doseₑₓₜₑᵣₙₐₗ) and the internal dose (doseᵢₙₜₑᵣₙₐₗ) for container 20 as follows:
doseₑₓₜₑᵣₙₐₗ/doseᵢₙₜ=1.16

Experiment C is performed using the same or a like container 20 having like properties. Container 20 in Experiment C is filled with the surrogate material, i.e., the dog food. Dosimeters are placed in like locations on the exterior of container 20 and dosimeters, together with the battery heater are placed within container 20 at the same location as Experiment B. In Experiment C, the battery-energized heater is not activated, i.e., not turned on, since the surrogate material is at ambient temperature. The heater assembly is included in container 20 in Experiment C to duplicate the presence of the heater assembly in container 20, as tested in Experiment B. Container 20 is irradiated with gamma rays at the same energy level as Experiment B. Radiation dose absorption levels are determined from the external dosimeters and the internal dosimeters. Experiment C establishes a numeric ratio between the external dose (doseₑₓₜₑᵣₙₐₗ) and the internal dose (doseᵢₙₜₑᵣₙₐₗ) for container 20 as follows:
doseₑₓₜₑᵣₙₐₗ/doseᵢₙₜₑᵣₙₐₗ=1.18

The results from Experiment B and Experiment C show less than a 2% difference, i.e., between the numeric ratio values when using dry ice and when using a surrogate material (dog food), under the same testing conditions. Experiments B and C basically indicate that the surrogate material mimics the physical properties of dry ice during irradiation.

Having established that the surrogate material simulated the properties of dry ice and could be used as a suitable surrogate for testing, Experiment D is conducted to establish a mathematical relationship between dose absorption at an external reference position on the external surface of container 20 and the dose absorption at an internal position within container 20. In Experiment D, container 20 is filled with the surrogate material and a dosimeter is placed on the external surface of container 20 at the reference position, and a dosimeter is placed within container 20 at the desired internal position, preferably the centermost location. The heater assembly is not within container 20, as in Experiments B and C. Elimination of the heater assembly eliminates any shielding effect the heater assembly may have on the actual dose absorbed within container 20.

Container 20, having a dosimeter on the external surface thereof, and the surrogate material and a dosimeter within the interior thereof, is then irradiated by gamma radiation. The absorbed doses at the two locations are then determined. A mathematical ratio for container 20 from the doseₑₓₜₑᵣₙₐₗ reference position to the doseᵢₙₜₑᵣₙₐₗ reference position is determined as follows:
doseₑₓₜₑᵣₙₐₗ/doseᵢₙₜₑᵣₙₐₗ=1.1

This mathematical ratio is confirmed using biological samples, Bacillus pumilus strips in simulated testing using gamma radiation. Bacillus pumilus strips with an initial population of 1.1×10⁶ having a D₁₀ value of 1.4 kGy are selected for testing. The objective of the simulated testing is to achieve a 6-log reduction of the population of the Bacillus pumilus and a 7-log reduction of the population of the Bacillus pumilus. A 6-log reduction will give one positive growth per Bacillus pumilus strip, and a 7-log reduction will give zero positive growths per Bacillus pumilus strip. The required dose to effect a 6-log reduction would be approximately 6×1.4 kGy, i.e., approximately 8.4 kGy, and the required dose to effect a 7-log reduction would be approximately 7×1.4 kGy, i.e., approximately 9.8 kGy.

A first series of tests are performed with Bacillus pumilus strips packed within the container within dry ice. Container 20 having the dry ice and Bacillus pumilus strips therein is irradiated. It is desired that the Bacillus pumilus strips within the dry ice within container 20 receive a radiation dose of 7 kGy. This desired dose to be applied to the internal Bacillus pumilus strips is adjusted using the aforementioned mathematical ratio of 1.1 to compensate for the shielding effect of the surrogate material and container 20. Accordingly, for the Bacillus pumilus strips within the dry ice within container 20 to receive a dose of 7 kGy, the dose is adjusted to 7.7 kGy, i.e., 7 kGy×1.1=7.7 kGy. In other words, it is believed that if 7.7 kGy is the applied dose as determined by external dosimeter 62, the internal dose applied to the Bacillus pumilus strips packed within the dry ice within container 20 should be 7 kGy. For statistical analysis, the foregoing test is repeated for identical containers 20 having Bacillus pumilus strips within dry ice at different dose levels. The desired doses to be applied to the Bacillus pumilus strips are 8 kGy, 9 kGy, 10 kGy and 11 kGy. Applying the aforementioned mathematical ratio of 1.1 to the foregoing doses, individual doses of 8.8 kGy, 9.9 kGy, 11 kGy and 12.1 kGy are applied to the external surfaces of containers 20 containing Bacillus pumilus strips.

A second series of tests on like containers 20 containing Bacillus pumilus strips packed within the surrogate material (i.e., the dog food) within containers 20 are also conducted under the similar conditions. In this respect, individual doses of 7.7 kGy, 8.8 kGy, 9.9 kGy, 11 kGy and 12.1 kGy are applied to separate, like containers 20 containing the Bacillus pumilus strips packed within the surrogate material.

It is believed that the dry ice will produce a cryo-preservation effect on the Bacillus pumilus. Therefore, three additional tests are conducted on the Bacillus pumilus under dry ice conditions. In this respect, tests to apply doses of 12 kGy, 13 kGy, and 14 kGy to the Bacillus pumilus are conducted. The desired application doses are adjusted by the aforementioned numeric ratio of 1.1 to 13.2 kGy, 14.2 kGy, and 15.4 kGy to compensate for the shielding effect of container 20 when testing the Bacillus pumilus.

Bacillus pumilus strips from the foregoing procedures are sent to a laboratory for sterility testing. The procedures for Fraction Negative and Limited Spearman-Karber testing are as follows: Ten biological indicators of each dose are individually transferred to tubes containing a growth medium, i.e., of soybean casein digest broth. The strips are incubated at 30° C. to 35° C. for seven (7) days then scored as positive or negative for growth.

The procedures for Survivor Curve testing are as follows: Three biological indicators of each dose are tested for population verification by pooling three biological indicators together, vortexing the strips in sterile water with sterile glass beads until macerated, performing serial dilutions, and plating onto soybean casein digest agar. The plates are incubated at 30° C. to 35° C. for two (2) days then enumerated.

The D₁₀ value of each organism is determined using the Limited Spearman-Karber, Fraction Negative, and Survivor Curve methods. The calculations are as follows:

Limited Spearman-Karber Testing

Details on performing this calculation may be found in ANSI/AAMI/ISO 11138, Annex D.

$$Fraction Negative \text{:} \frac{dose}{log {N}_{0} - log MPN}$$

where:

N₀=Number of organisms on biological indicator pre-irradiation

MPN=In (# biological indicators tested/# biological indicators negative)

$$Survivor Curve \text{:} \frac{dose}{log {N}_{0} - log {N}_{f}}$$

where:

N₀=Number of organisms on biological indicator pre-irradiation

N_{f}=Number of organisms on biological indicator post-irradiation

For the Limited Spearman-Karber calculation, it is necessary to use doses with exact intervals in the calculations (e.g. 9.0, 10.0 kGy) rather than the actual doses delivered (e.g. 9.1, 10.3 kGy). This is a restriction required by the calculation and cannot be avoided.

The results of sterility and population verification testing are as follows:

| | 9.1 | 10.3 | 11.1 | 12.1 | 13.5 | 14.4 | 15.6 |
|---|---|---|---|---|---|---|---|
| Dry lce | kGy | kGy | kGy | kGy | kGy | kGy | kGy |
| # Negative | 0 | 0 | 0 | 1 | 3 | 10 | 10 |
| CFU/Strip | UFA | ˜17¹ | UFA | UFA | UFA | UFA | UFA |

| | 8.3 | 9.5 | 10.7 | 11.8 | 12.8 |
|---|---|---|---|---|---|
| Dog Food | kGy | kGy | kGy | kGy | kGy |
| # Negative | 0 | 1 | 4 | 10 | 10 |
| CFU/Strip | ˜1.1² | UFA | UFA | UFA | UFA |

Number of strips negative for growth out of 10 tested and colony forming units (CFU) per strip after receiving the specified dose in kGy.
UFA = results that are outside of the statistically accurate range for a plate count.
¹In this case even the 10.3 kGy results are below the desired range. This dose is used for the D₁₀ value determination because it is the lowest dose, which resulted in any growth due to the dilutions used for testing the 8.8 kGy strips.
²In this case even the 8.3 kGy results are below the desired range. This dose is used for the D₁₀ value determination because it is the lowest dose, which resulted in any growth from the strips.

Summary Table - Calculated D₁₀ values for Dry Ice and Dog Food

| | Fraction | Survivor | Limited | Limited SK |
|---|---|---|---|---|
| | Negative | Curve | SK | 95% Confidence |
| Dry Ice | 2.20 kGy | 2.14 kGy | 2.13 kGy | 2.07-2.19 kGy |
| Dog Food | 1.72 kGy | 1.38 kGy | 1.68 kGy | 1.62-1.75 kGy |

In general, this series of experiments demonstrates that irradiation of materials in the configuration described above provides a reliable method of determining an external monitoring position and corresponding dose ratio to use in microbial reduction of low temperature samples. When using the established numeric dose ratio discussed above, the ambient temperature spore strips (the biological indicators irradiated in dog food surrogate) accurately reflect the expected D₁₀ value. Spore strips irradiated in the frozen state exhibit a measurably higher D₁₀ value. This is consistent with the cryo-preservative effects of low temperatures on biological systems. If tissue is irradiated in a frozen state to preserve cellular qualities, it follows that bacteria will also benefit from this cryo-preservative effect. This difference in D₁₀ value is an interesting result of the experimentation, but is independent of the ratio establishment and does not exclude using surrogate material for dose mapping.

Using surrogate material data and biological spore strips data, the present invention provides a procedure for establishing a dose ratio with a standard container 20, which procedure provides a useful method of quickly and accurately determining the irradiation dose to frozen material, i.e., tissue specimens. The present invention thus provides a method of determining the dose of irradiation applied to a frozen material without invading the integrity of the frozen material.

The present invention may be further understood by reference to the attached article entitled: “IMPROVED METHOD FOR GAMMA IRRADIATION OF DONOR TISSUE” by STERIS Isomedix Services, which is incorporated herein by reference.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

1. A method for determining a dose of radiation applied to a frozen material, comprising the steps of:
irradiating a frozen material in a container;
determining an applied dose of radiation applied to a first location on said container; and
determining an absorbed dose of radiation for the frozen material at a second location within said container in accordance with predetermined data relating to said container.

2. A method as defined in claim 1, wherein said irradiating step is carried out for a predetermined period of time.

3. A method as defined in claim 1, wherein said container has predetermined insulating properties when said container contains dry ice.

4. A method as defined in claim 3, wherein determining the insulating properties of said container when said container contains dry ice comprises:
placing dry ice within said container;
monitoring an external surface temperature of said container for a predetermined period of time; and
determining whether said external surface temperature dropped below a predetermined minimum temperature.

5. A method as defined in claim 1, wherein said predetermined data relating to said container comprises a mathematical ratio.

6. A method as defined in claim 5, wherein said mathematical ratio is determined by:
using a surrogate material in place of dry ice during irradiation testing;
establishing a delivered dose ratio for said container having said surrogate material therein; and
conducting biological testing to verify the validity of said delivered dose ratio.

7. A method as defined in claim 6, wherein a surrogate material to be used in place of dry ice during irradiation testing is determined by the steps of:
obtaining a first dose absorption profile of a container whereby said container contains a surrogate material for dry ice;
obtaining a second dose absorption profile of said container whereby said container contains dry ice;
comparing said first dose absorption profile with said second dose absorption profile;
determining a difference between said first dose absorption profile and said second dose absorption profile; and
comparing said difference between said first dose absorption profile and said second dose absorption profile with a predetermined limit.

8. A method as defined in claim 7, wherein the step for obtaining a first dose absorption profile of said container containing a surrogate material comprises the steps of:
placing a surrogate material for dry ice within said container and placing a first dosimeter at said first location and placing a second dosimeter at said second location;
irradiating said container;
determining a dosage of radiation received at said first dosimeter; and
determining a dosage of radiation received at said second dosimeter.

9. A method as defined in claim 8, wherein said container includes a geometric center, said second location being at said geometric center of said container.

10. A method as defined in claim 7, wherein the step for obtaining a second dose absorption profile of said container whereby said container contains dry ice comprises the steps of:
placing dry ice within said container and placing a first dosimeter at said first location and placing a second dosimeter at said second location;
irradiating said container;
determining a dosage of radiation received at said first dosimeter; and
determining a dosage of radiation received at said second dosimeter.

11. A method as defined in claim 10, wherein said container includes a geometric center, said second location being at said geometric center of said container.

12. A method as defined in claim 6, wherein said step of establishing a delivered dose ratio for said container comprises the steps of:
using said surrogate material to surround a target material located within said container;
exposing said container having said surrogate material therein to a radiation source;
determining a radiation dosage received at said first location on said container from said radiation source;
determining a radiation dosage received at said second location within said container from said radiation source; and
dividing said radiation dosage received at said first location by said radiation dosage received at said second location.

13. A method as defined in claim 6, wherein said step for conducting biological testing to verify the validity of said delivered dose ratio comprises the steps of:
packing biological test material into a container having dry ice therein;
irradiating said container having said biological test material and said dry ice therein wherein said irradiating is sufficient to deliver a desired dose of radiation to said biological test material;
measuring amount of said radiation received at said first location;
delivering sufficient radiation to said first location such that a desired amount of radiation is delivered to said biological test material wherein said sufficient radiation to said first location is determined by multiplying said desired amount of radiation by said dosage ratio; and
testing said biological test material to determine the efficacy of radiation delivered to said biological test material.

14. A method as defined in claim 6, wherein said surrogate material for dry ice is dog food.
